(19) United States
(12) Patent Application Publication
(10) Pub. No.: US 2008/0132944 A1
(43) Pub. Date: Jun. 5, 2008

(54) Suture re-tensioning device

(76) Inventor: Donald Walter Kress, Frederick, MD (US)

Correspondence Address: MANELLI DENISON & SELTER, 2000 M STREET NW SUITE 700, WASHINGTON, DC, 20036-3307, US

(21) Appl. No.: 11/606,919

(22) Filed: Dec. 1, 2006

(51) Int. Cl. A61B 17/04

(52) U.S. Cl. 606/228; 606/232

(57) ABSTRACT

A suture re-tensioning device 10 includes a body 12 having an opening 14 constructed and arranged to permit a portion of a surgical suture 16 to pass there-through. The body is constructed and arranged to be implantable in a human. Retaining structure 18 is associated with the body and is constructed and arranged to 1) permit the suture to be moved through the opening in a certain direction so as to control tension of the suture and 2) secure the suture with respect to the body so as to prevent the suture from moving in a direction opposite the certain direction yet permitting further movement of the suture in the certain direction.

### FIELD OF THE INVENTION

This invention relates to the field of Plastic Surgery and, more particularly, to a device for re-tensioning sutures used in such surgery.

### BACKGROUND OF THE INVENTION

As the field of Plastic Surgery moves in the direction of less invasive procedures, a variety of different suture materials have emerged which have the ability to lift the tissues and suspend them for variable periods of time. Some examples of procedures which rely on lifting of the tissues include: Neck lift; Forehead lift; Face lift and mini face lift (Life Style Lift, Quick lift, S-Lift, and MACS Lift); Brow lift; Breast Lift; Buttock Lift; Thigh Lift; and others.

All of the above-mentioned procedures have in common some form of proximal anchoring system to bone, fascia (temporal, mastoid, or Galea to name a few), or other supporting tissues. The lifting mechanism is then placed in the mobile tissues and the lift is effected. The lifting mechanism can include various types of suture material which may be smooth, or knotted with cones (Silhouette), or barbed (Woffles lift, Aptos, Contour). Once this lifting mechanism is deployed and attached to the anchor, several predictable things begin to happen. At the point of the most stress, there is cell death and a gradual relaxation of the overall lift effect. There is also remodeling and relaxation of the involved tissues which also contributes to premature failure of the lifting mechanism.

Attempts have been made in the past to untie and retie the suture materials in an effort to regain the lost lift effect after it becomes apparent. Largely, this technique has been ineffective because of the mechanical difficulty of untying a knot and the damage this does to the tensile strength of the suture.

Thus, there is a need to provide an implantable device that can re-tension the sutures and reestablish their effectiveness.

### SUMMARY OF THE INVENTION

An object of the invention is to fulfill the need referred to above. In accordance with the principles of the present invention, this objective is achieved by providing a suture re-tensioning device including a body having an opening constructed and arranged to permit a portion of a surgical suture to pass there-through. The body is constructed and arranged to be implantable in a human. The device includes retaining structure associated with the body an constructed and arranged to 1) permit the suture to be moved through the opening in a certain direction so as to control tension of the suture and 2) secure the suture with respect to the body so as to prevent the suture from moving in a direction opposite the certain direction yet permitting further movement of the suture in the certain direction.

In accordance with another aspect of the invention, a method of adjusting tension of sutures includes implanting and securing at least one suture re-tensioning device in a human. The device includes a body with an opening constructed and arranged to permit a surgical suture to pass there-through and retaining structure associated with the body. A first portion of a suture is moved through the opening in the device, with a second portion of the suture being secured to portions of human tissue to suspend the tissue. A certain tension is established in the suture by further moving the first portion of the suture through the opening in one direction and engaging the first portion of the suture with the retaining structure, thereby securing the first portion of the suture with respect to the body at the certain tension and preventing the suture from moving in a direction opposite the certain direction. If the tension of the suture becomes less than the certain tension, the method further includes moving the first portion of the suture further through the opening in the one direction and reengaging the first portion of the suture with the retaining structure to substantially approach or achieve the certain tension.

Other objects, features and characteristics of the present invention, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawing, wherein like reference numerals refer to like parts, in which:

FIG. 1 is an enlarged, cross-sectional view of a suture re-tensioning device shown engaging a suture in accordance with the principles of the invention.

FIG. 2 is an enlarged view of suture retaining structure of the re-tensioning device of FIG. 1.

FIG. 3 is a side view of a patient employing the device of FIG. 1 in a mini-face lift procedure.

FIG. 4 is a front view of a patient showing another embodiment of the anchor structure.

FIG. 5 is an enlarged, cross-sectional view of another embodiment of a suture re-tensioning device shown engaging a pair of sutures.

### DETAILED DESCRIPTION OF THE EXEMPLARY EMBODIMENT

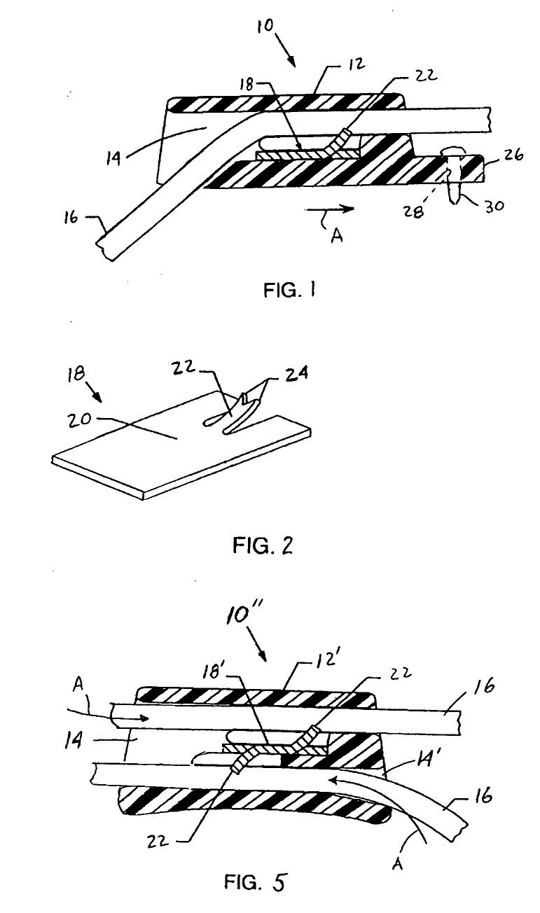

With reference to FIG. 1, a suture re-tensioning device is shown, generally indicated at 10, in accordance with an embodiment of the present invention. The device 10 includes a body 12 having at least one opening 14 constructed and arranged to permit a portion of a surgical suture 16 to pass there-through. The body 12 is constructed and arranged to be implantable in a human and is of suitable material such as a surgical grade polymer, for example, polyethylene or polyetheretherketone (PEEK) polymer.

The device 10 includes suture retaining structure generally indicated at 18 in FIG. 1. In the embodiment and as best shown in FIG. 2, the suture retaining structure has a body 20 that is preferably in the form of a plate stamped from surgical grade spring steel. The structure 18 includes spring structure preferably in the form an upwardly angled barb 22. The barb 22 may have a straight edge but preferably has one or more upwardly and forwardly extending teeth 24. The barb 22 defines a cantilevered spring extending in the opening 14. The structure 18 can be constructed from other materials suitably compatible with implantation into the human body. Thus, as will be described more fully below, the structure 18 permits a suture to be moved through the opening 14 in a certain direction (e.g. rearwardly or in direction A in FIG. 1) so as to control tension of the suture and secures the suture with respect to the body 12, with the barb 22 preventing the suture from moving in a direction opposite the certain direction, yet permits further movement of the suture in the certain direction.

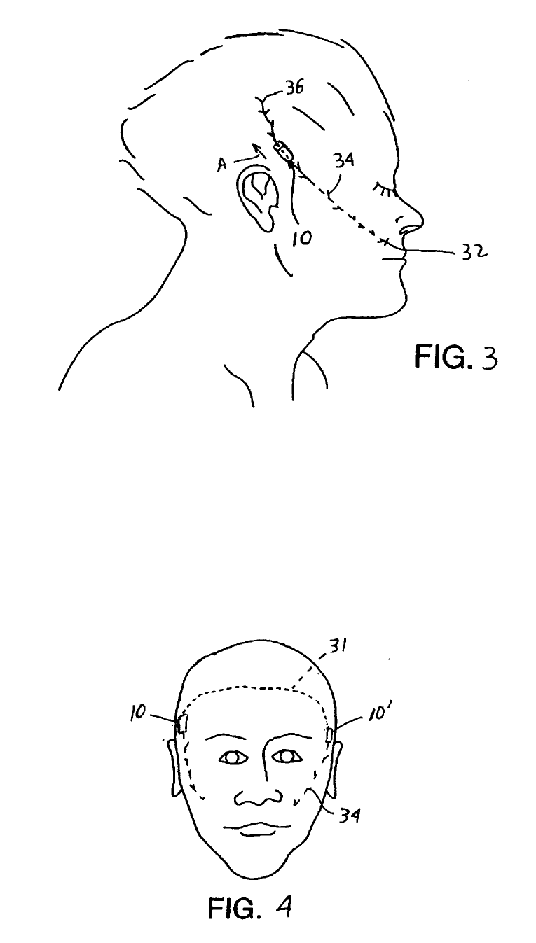

The body 12 preferably includes, or has in combination therewith, anchor structure to secure the device 10 with respect to human tissue. In the illustrated embodiment, the anchor structure includes a tab 26 having a bore 28 for receiving a fastener 30 or the like to secure the device 20 to bone (FIG. 3). Instead of using a fastener, the tab 26 or other portion of the body 12 can be secured to fascia (e.g., temporal, mastoid, or Galea) with a suture. Alternatively, the anchor structure can include a Dacron patch constructed and arranged to secure the body 12 with respect to human tissue. Still further, and with reference to FIG. 4, first and second suture retaining devices can be provided with the first device 10 being implanted one side of a human face and the second device 10′ being implanted on the opposite side of face. The first device 10 can be secured to the second device 10 by coupling a joining suture 31 to the first device 10, extending the suture 31 under the scalp, and coupling the suture 31 to the second device 10′.

With reference to FIG. 3, during a mini-lift procedure, a surgeon secures a proximal end 32 of a suture 24 under the patient's skin. The suture 24 may be may be smooth, but is preferably knotted with cones or is barbed. The re-tensioning device 10 is implanted and secured to bone or fascia of the patient. A distal end 36 of the suture 34 is passed through the opening 14 (FIG. 1) of the suture re-tensioning device 10 to the appropriate tension for lifting tissue, and the surgeon releases the suture 34. Thus, the spring or barb 22 of the device 10 permits the suture 34 to pass easily through the opening 14 in direction A, but resiliently engages the suture 34 and prevents the suture 34 from moving in a direction opposite direction A. If, after time, the surgeon determines that the tension of the suture 34 has relaxed thus, permitting the lifted tissue to sag, the surgeon can again access the distal end 36 of the suture 34 and move the suture 34 further in the direction A to re-tension the suture and thus return (or substantially return) the lifted tissue to its proper position. Hence the device 10 advantageously maintains (adjusts) the mini face-lift or other tissue lifting procedures due to the ability of re-tensioning the sutures, instead of having to untie and re-tie sutures or totally re-perform the procedure if the tension of the sutures relaxes.

FIG. 5 shows another embodiment of the suture retaining structure 10″. As shown, the body 12′ of the device 10″ includes two openings 14, 14′, each receiving an associated suture 16. The retaining structure 18′ includes a barb 22 associated with a suture 16 permitting the associated suture to be adjustably moved through its opening in the direction A and secures the suture with respect to the body 12′ so as to prevent the suture from moving in a direction opposite the direction A.

Although a single spring or barb 22 has been shown to retain the suture 16, more than one barb or spring structure can be used to engage a single suture 16. For example, a spring or barb can engage opposing sides of a suture. Furthermore, as an alternative to the barb 22 or spring structure, the retaining structure can employ the principles of a “Chinese finger trap” whereby the body 12 can include a tube which grips the suture 16 passing there-through. The tube can comprise two or more layers of wires wrapped around the suture in helical fashion, wherein alternating layers have opposite hand. Still further, retaining structure can be of the cable tie technology such as that disclosed in U.S. Pat. No. 3,965,538, the contents of which is hereby incorporated by reference in its entirety into the present specification.

Thus, it can be appreciated that the retaining structure can include any structure that permits movement of a suture in one direction so as to adjust the tension thereof, yet secures and prevents the suture from moving in the opposite direction.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. A suture re-tensioning device comprising:
a body having an opening constructed and arranged to permit a portion of a surgical suture to pass there-through, the body being constructed and arranged to be implantable in a human, and
retaining structure associated with the body and constructed and arranged to 1) permit the suture to be moved through the opening in a certain direction so as to control tension of the suture and 2) secure the suture with respect to the body so as to prevent the suture from moving in a direction opposite the certain direction yet permitting further movement of the suture in the certain direction.

2. The device according to claim 1, wherein the body further includes anchor structure constructed and arranged to anchor the body with respect to human tissue.

3. The device of claim 2, wherein the anchor structure is a tab extending from the body.

4. The device of claim 3, wherein the human tissue is a bone and the device further comprises a fastener associated with the tab to secure the tab and thus the body to the bone.

5. The device according to claim 1, wherein the body is composed of surgical grade polymer.

6. The device of claim 1, wherein the retaining structure includes spring structure extending within the opening.

7. The device of claim 6, wherein the spring structure includes at least one cantilevered spring having a free end extending within the opening constructed and arranged to releasably engage the suture.

8. The device of claim 7, wherein the free end includes teeth extending in the direction opposite the certain direction.

9. The device of claim 1, wherein the retaining structure is composed of surgical grade spring steel.

10. A method of adjusting tension of sutures comprising:
implanting and securing at least one suture re-tensioning device in a human, the device comprising a body with an opening constructed and arranged to permit a surgical suture to pass there-through, and retaining structure associated with the body,
moving a first portion of a suture through the opening in the device, with a second portion of the suture being secured to portions of human tissue to suspend the tissue, and
establishing a certain tension in the suture by further moving the first portion of the suture through the opening in one direction and engaging the first portion of the suture with the retaining structure, thereby securing the first portion of the suture with respect to the body at the certain tension and preventing the suture from moving in a direction opposite the certain direction.

11. The method of claim 1, wherein, if the tension of the suture becomes less than the certain tension, the method further includes moving the first portion of the suture further through the opening in the one direction and reengaging the first portion of the suture with the retaining structure to substantially approach or achieve the certain tension.

12. The method of claim 10, wherein the step of implanting and securing includes providing first and second suture retaining devices and implanting the first device on one side of a human face and the second device on the opposite side of face, the method including securing the first device to the second device by coupling a joining suture to the first device and extending the joining suture under the scalp and coupling the joining suture to the second device.

13. The method of claim 10, wherein the step of securing includes using a fastener to secure the body to bone.

14. The method of claim 10, wherein the step of securing includes using a suture to secure the body to fascia.

15. The method of claim 10, wherein the step of securing includes using a Dacron patch to secure the body to tissue.

16. A suture re-tensioning device comprising:
a body having an opening constructed and arranged to permit a portion of a surgical suture to pass there-through, the body being constructed and arranged to be implantable in a human, and means, associated with the body, for 1) permitting the suture to be moved through the opening in a certain direction so as to control tension of the suture and 2) securing the suture with respect to the body so as to prevent the suture from moving in a direction opposite the certain direction yet permitting further movement of the suture in the certain direction.

17. The device according to claim 16, wherein the body further includes means for anchoring the body with respect to human tissue.

18. The device of claim 17, wherein the means for anchoring a tab extending from the body, wherein the human tissue is a bone and the device further comprises a fastener associated with the tab to secure the tab and thus the body to the bone.

19. The device of claim 16, wherein the means for permitting and securing includes spring structure extending within the opening.

20. The device of claim 16, in combination with a suture secured by the means for permitting and securing.
